(19) United States
(12) Patent Application Publication
(10) Pub. No.: US 2009/0227690 A1
(43) Pub. Date: Sep. 10, 2009

(54) Cyclodextrin Inclusion Complexes and Methods of Preparing Same

(75) Inventors: Kenneth J. Strassburger, Cincinnati, OH (US); Jacques Mazoyer, Baupte (FR)

Correspondence Address: MICHAEL BEST & FRIEDRICH LLP, 100 E WISCONSIN AVENUE, Suite 3300, MILWAUKEE, WI, 53202, US

(73) Assignee: CARGILL, INCORPORATED, Wayzata, MN (US)

(21) Appl. No.: 11/575,715

(22) PCT Filed: Nov. 1, 2004
(86) PCT No.: PCT/US04/36270

§ 371 Date: Nov. 26, 2008

Related U.S. Application Data
(60) Provisional application No. 60/613,261, filed on Sep. 27, 2004.

(51) Int. Cl. A61K 47/40; A61K 8/73; A61Q 13/00

(52) U.S. Cl. 514/777; 512/4

(57) ABSTRACT

Cyclodextrin inclusion complexes and methods for preparing cyclodextrin inclusion complexes. In some embodiments, the method for preparing a cyclodextrin inclusion complex may include dry blending cyclodextrin and an emulsifier to form a dry blend, and combining a solvent and a guest with the dry blend to form a cyclodextrin inclusion complex. In some embodiments, the method for preparing a cyclodextrin inclusion complex may include combining cyclodextrin and an emulsifier to form a first mixture, combining the first mixture with a solvent to form a second mixture, and combining a guest with the second mixture to form a third mixture.

### BACKGROUND OF THE INVENTION

The following U.S. patents disclose the use of cyclodextrins to complex various guest molecules, and are hereby fully incorporated herein by reference: U.S. Pat. Nos. 4,296,137, 4,296,138 and 4,348,416 to Borden (flavoring material for use in chewing gum, dentifrices, cosmetics, etc.); 4,265,779 to Gandolfo et al. (suds suppressors in detergent compositions); 3,816,393 and 4,054,736 to Hyashi et al. (prostaglandins for use as a pharmaceutical); 3,846,551 to Mifune et al. (insecticidal and acaricidal compositions); 4,024,223 to Noda et al. (menthol, methyl salicylate, and the like); 4,073,931 to Akito et al. (nitro-glycerine); 4,228,160 to Szjetli et al. (indomethacin); 4,247,535 to Bernstein et al. (complement inhibitors); 4,268,501 to Kawamura et al. (anti-asthmatic actives); 4,365,061 to Szjetli et al. (strong inorganic acid complexes); 4,371,673 to Pitha (retinoids); 4,380,626 to Szjetli et al. (hormonal plant growth regulator), 4,438,106 to Wagu et al. (long chain fatty acids useful to reduce cholesterol); 4,474,822 to Sato et al. (tea essence complexes); 4,529,608 to Szjetli et al. (honey aroma), 4,547,365 to Kuno et al. (hair waving active-complexes); 4,596,795 to Pitha (sex hormones); 4,616,008 Hirai et al. (antibacterial complexes); 4,636,343 to Shibanai (insecticide complexes), 4,663,316 to Ninger et al. (antibiotics); 4,675,395 to Fukazawa et al. (hinokitiol); 4,732,759 and 4,728,510 to Shibanai et al. (bath additives); 4,751,095 to Karl et al. (aspartamane); 4,560,571 (coffee extract); 4,632,832 to Okonogi et al. (instant creaming powder); 5,571,782, 5,660,845 and 5,635,238 to Trinh et al. (perfumes, flavors, and pharmaceuticals); 4,548,811 to Kubo et al. (waving lotion); 6,287,603 to Prasad et al. (perfumes, flavors, and pharmaceuticals); 4,906,488 to Pera (olfactants, flavors, medicaments, and pesticides); and 6,638,557 to Qi et al. (fish oils).

Cyclodextrins are further described in the following publications, which are also incorporated herein by reference: (1) Reineccius, T. A., et al. “Encapsulation of flavors using cyclodextrins: comparison of flavor retention in alpha, beta, and gamma types.” Journal of Food Science. 2002; 67(9): 3271-3279; (2) Shiga, H., et al. “Flavor encapsulation and release characteristics of spray-dried powder by the blended encapsulant of cyclodextrin and gum arabic.” Marcel Dekker, Incl., www.dekker.com. 2001; (3) Szente L., et al. “Molecular Encapsulation of Natural and Synthetic Coffee Flavor with β-cyclodextrin.” Journal of Food Science. 1986; 51(4): 1024-1027; (4) Reineccius, G. A., et al. “Encapsulation of Artificial Flavors by β-cyclodextrin.” Perfumer & Flavorist (ISSN 0272-2666) An Allured Publication. 1986: 11(4): 2-6; and (5) Bhandari, B. R., et al. “Encapsulation of lemon oil by paste method using β-cyclodextrin: encapsulation efficiency and profile of oil volatiles.” J. Agric. Food Chem. 1999; 47: 5194-5197.

### SUMMARY OF THE INVENTION

Some embodiments of the present invention provide a method for preparing a cyclodextrin inclusion complex. The method can include dry blending cyclodextrin and an emulsifier to form a dry blend, and combining a solvent and a guest with the dry blend to form a cyclodextrin inclusion complex.

In some embodiments of the present invention, a method for preparing a cyclodextrin inclusion complex is provided. The method can include combining cyclodextrin and an emulsifier to form a first mixture, combining the first mixture with a solvent to form a second mixture, and combining a guest with the second mixture to form a third mixture.

Some embodiments of the present invention provide a method for preparing a cyclodextrin inclusion complex. The method can include dry blending cyclodextrin and pectin to form a first mixture, combining the first mixture with water to form a second mixture, and combining diacetyl with the second mixture to form a third mixture.

Other features and aspects of the invention will become apparent to those skilled in the art upon review of the following detailed description, claims and drawings.

### BRIEF DESCRIPTION OF THE DRAWINGS

FIG. 1 is a schematic illustration of a cyclodextrin molecule having a cavity, and a guest molecule held within the cavity.

FIG. 2 is a schematic illustration of a nano-structure formed by self-assembled cyclodextrin molecules and guest molecules.

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways. Also, it is to be understood that the phraseology and terminology used herein is for the purpose of description and should not be regarded as limited. The use of “including,” “comprising” or “having” and variations thereof herein is meant to encompass the items listed thereafter and equivalents thereof as well as additional items.

### DETAILED DESCRIPTION

The present invention is generally directed to cyclodextrin inclusion complexes and methods of forming them. Some cyclodextrin inclusion complexes of the present invention provide for the encapsulation of volatile and reactive guest molecules. In some embodiments, the encapsulation of the guest molecule can provide at least one of the following: (1) prevention of a volatile or reactive guest from escaping a commercial product which may result in a lack of flavor intensity in the commercial product; (2) isolation of the guest molecule from interaction and reaction with other components that would cause off note formation; (3) stabilization of the guest molecule against degradation (e.g., hydrolysis, oxidation, etc.); (4) selective extraction of the guest molecule from other products or compounds; (5) enhancement of the water solubility of the guest molecule; (6) taste or odor improvement or enhancement of a commercial product; (7) thermal protection of the guest in a microwave and conventional baking applications; (8) slow and/or sustained release of flavor or odor (e.g., in embodiments employing diacetyl as the guest molecule in cyclodextrin inclusion complex, it can provide the perception of melting butter); and (9) safe handling of guest molecules.

As used herein, the term “cyclodextrin” can refer to a cyclic dextrin molecule that is formed by enzyme conversion of starch. Specific enzymes, e.g., various forms of cycloglycosyltransferase (CGTase), can break down helical structures that occur in starch to form specific cyclodextrin molecules having three-dimensional polyglucose rings with, e.g., 6, 7, or 8 glucose molecules. For example, α-CGTase can convert starch to o-cyclodextrin having 6 glucose units, β-CGTase can convert starch to β-cyclodextrin having 7 glucose units, and γ-CGTase can convert starch to γ-cyclodextrin having 8 glucose units. Cyclodextrins include, but are not limited to, at least one of α-cyclodextrin, β-cyclodextrin, γ-cyclodextrin, and combinations thereof.

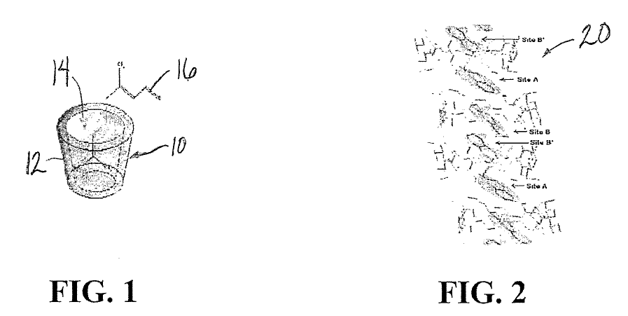

The three-dimensional cyclic structure (i.e., macrocyclic structure) of a cyclodextrin molecule 10 is shown schematically in FIG. 1. The cyclodextrin molecule 10 includes an external portion 12, which includes primary and secondary hydroxyl groups, and which is hydrophilic. The cyclodextrin molecule 10 also includes a three-dimensional cavity 14, which includes carbon atoms, hydrogen atoms and ether linkages, and which is hydrophobic. The hydrophobic cavity 14 of the cyclodextrin molecule can act as a host and hold a variety of molecules, or guests 16, that include a hydrophobic portion to form a cyclodextrin inclusion complex.

As used herein, the term “guest” can refer to any molecule of which at least a portion can be held or captured within the three dimensional cavity present in the cyclodextrin molecule, including, without limitation, at least one of a flavor, an olfactant, a pharmaceutical agent, a nutraceutical agent, and combinations thereof.

Examples of flavors can include, without limitation, flavors based on aldehydes, ketones or alcohols. Examples of aldehyde flavors can include, without limitation, at least one of: acetaldehyde (apple); benzaldehyde (cherry, almond); anisic aldehyde (licorice, anise); cinnamic aldehyde (cinnamon); citral, i.e. alpha citral (lemon, lime); neral, i.e. beta citral (lemon, lime); decanal (orange, lemon); ethyl vanillin (vanilla, cream); heliotropine, i.e. piperonal (vanilla, cream); vanillin (vanilla, cream); a-amyl cimiamaldehyde (spicy fruity flavors); butyraldehyde (butter, cheese); valeraldehyde (butter, cheese); citronellal (modifies, many types); decenal (citrus fruits); aldehyde C-8 (citrus fruits); aldehyde C-9 (citrus fruits); aldehyde C-12 (citrus fruits); 2-ethyl butyraldehyde (berry fruits); hexenal, i.e. trans-2 (berry fruits); tolyl aldehyde (cherry, almond); veratraldehyde (vanilla); 2-6-dimethyl-5-heptenal, i.e. Melonal™ (melon); 2,6-dimethyloctanal (green fruit); 2-dodecenal (citrus, mandarin); and combinations thereof.

Examples of ketone flavors can include, without limitation, at least one of: d-carvone (caraway); l-carvone (spearmint); diacetyl (butter, cheese, “cream”); benzophenone (fruity and spicy flavors, vanilla); methyl ethyl ketone (berry fruits); maltol (berry fruits) menthone (mints), methyl amyl ketone, ethyl butyl ketone, dipropyl ketone, methyl hexyl ketone, ethyl amyl ketone (berry fruits, stone fruits); pyruvic acid (smokey, nutty flavors); acetanisole (hawthorn heliotrope); dihydrocarvone (spearmint); 2,4-dimethylacetophenone (peppermint); 1,3-diphenyl-2-propanone (almond); acetocumene (orris and basil, spicy); isojasmone (jasmine); d-isomethylionone (orris like, violet); isobutyl acetoacetate (brandy-like); zingerone (ginger); pulegone (peppermint-camphor); d-piperitone (minty); 2-nonanone (rose and tea-like); and combinations thereof.

Examples of alcohol flavors can include, without limitation, at least one of anisic alcohol or p-methoxybenzyl alcohol (fruity, peach); benzyl alcohol (fruity); carvacrol or 2-p-cymenol (pungent warm odor); carveol; cinnamyl alcohol (floral odor); citronellol (rose like); decanol; dihydrocarveol (spicy, peppery); tetrahydrogeraniol or 3,7-dimethyl-1-octanol (rose odor); eugenol (clove); p-mentha-1,8dien-7-Oλ or perillyl alcohol (floral-pine); and combinations thereof.

Examples of olfactants can include, without limitation, at least one of natural fragrances, synthetic fragrances, synthetic essential oils, natural essential oils, and combinations thereof.

Examples of the synthetic fragrances can include, without limitation, at least one of terpenic hydrocarbons, esters, ethers, alcohols, aldehydes, phenols, ketones, acetals, oximes, and combinations thereof.

Examples of terpenic hydrocarbons can include, without limitation, at least one of lime terpene, lemon terpene, limonen dimer, and combinations thereof.

Examples of esters can include, without limitation, at least one of γ-undecalactone, ethyl methyl phenyl glycidate, allyl caproate, amyl salicylate, amyl benzoate, amyl acetate, benzyl acetate, benzyl benzoate, benzyl salicylate, benzyl propionate, butyl acetate, benzyl butyrate, benzyl phenylacetate, cedryl acetate, citronellyl acetate, citronellyl formate, p-cresyl acetate, 2-t-pentyl-cyclohexyl acetate, cyclohexyl acetate, cis-3-hexenyl acetate, cis-3-hexenyl salicylate, dimethylbenzyl acetate, diethyl phthalate, δ-deca-lactone dibutyl phthalate, ethyl butyrate, ethyl acetate, ethyl benzoate, fenchyl acetate, geranyl acetate, γ-dodecalatone, methyl dihydrojasmonate, isobornyl acetate, β-isopropoxyethyl salicylate, linalyl acetate, methyl benzoate, o-t-butylcylohexyl acetate, methyl salicylate, ethylene brassylate, ethylene dodecanoate, methyl phenyl acetate, phenylethyl isobutyrate, phenylethylphenyl acetate, phenylethyl acetate, methyl phenyl carbinyl acetate, 3,5,5-trimethylhexyl acetate, terpinyl acetate, triethyl citrate, p-t-butylcyclohexyl acetate, vetiver acetate, and combinations thereof.

Examples of ethers can include, without limitation, at least one of p-cresyl methyl ether, diphenyl ether, 1,3,4,6,7,8-hexahydro-4,6,7,8,8-hexamethyl cyclopenta-β-2-benzopyran, phenyl isoamyl ether, and combinations thereof.

Examples of alcohols can include, without limitation, at least one of n-octyl alcohol, n-nonyl alcohol, β-phenylethyldimethyl carbinol, dimethyl benzyl carbinol, carbitol dihydromyrcenol, dimethyl octanol, hexylene glycol linalool, leaf alcohol, nerol, phenoxyethanol, γ-phenyl-propyl alcohol, β-phenylethyl alcohol, methylphenyl carbinol, terpineol, tetraphydroalloocimenol, tetrahydrolinalool, 9-decen-1-ol, and combinations thereof.

Examples of aldehydes can include, without limitation, at least one of n-nonyl aldehyde, undecylene aldehyde, methylnonyl acetaldehyde, anisaldehyde, benzaldehyde, cyclamenaldehyde, 2-hexylhexanal, ahexylcinnamic alehyde, phenyl acetaldehyde, 4-(4-hydroxy-4-methylpentyl)-3-cyclohexene-1-carboxyaldehyde, p-t-butyl-a-methylhydro-cinnamic aldehyde, hydroxycitronellal, α-amylcinnamic aldehyde, 3,5-dimethyl-3-cyclohexene-1-carboxyaldehyde, and combinations thereof.

Examples of phenols can include, without limitation, methyl eugenol.

Examples of ketones can include, without limitation, at least one of l-carvone, α-damascon, ionone, 4-t-pentylcyclohexanone, 3-amyl-4-acetoxytetrahydropyran, menthone, methylionone, p-t-amycyclohexanone, acetyl cedrene, and combinations thereof.

Examples of the acetals can include, without limitation, phenylacetaldehydedimethyl acetal.

Examples of oximes can include, without limitation, 5-methyl-3-heptanon oxime.

A guest can further include, without limitation, at least one of fatty acids, lactones, terpenes, diacetyl, dimethyl sulfide, proline, furaneol, linalool, acetyl propionyl, natural essences (e.g., orange, tomato, apple, cinnamon, raspberry, etc.), essential oils (e.g., orange, lemon, lime, etc.), and combinations thereof.

As used herein, the term “cyclodextrin inclusion complex” refers to a complex that is formed by encapsulating at least a portion of one or more guest molecules with one or more cyclodextrin molecules (encapsulation on a molecular level) by capturing and holding a guest molecule within the three dimensional cavity. The guest can be held in position by van der Waal forces within the cavity by at least one of hydrogen bonding and hydrophilic-hydrophobic interactions. The guest can be released from the cavity when the cyclodextrin inclusion complex is dissolved in water.

As used herein, the term “hydrocolloid” generally refers to a substance that forms a gel with water. A hydrocolloid can include, without limitation, at least one of xanthan gum, pectin, gum arabic (or gum acacia), tragacanth, guar, carrageenan, locust bean, and combinations thereof.

As used herein, the term “pectin” refers to a hydrocolloidal polysaccharide that can occur in plant tissues (e.g., in ripe fruits and vegetables). Pectin can include, without limitation, at least one of beet pectin, fruit pectin (e.g., from citrus peels), and combinations thereof. The pectin employed can be of varying molecular weight.

Cyclodextrin inclusion complexes of the present invention can be used in a variety of applications, including, without limitation, at least one of foods (e.g., popcorn, cereal, coffee, cookies, brownies, other baked goods, etc.), chewing gums, candy, flavorings, fragrances, pharmaceuticals, nutraceuticals, cosmetics, agricultural applications (e.g., herbicides, pesticides, etc.), photographic emulsions, and combinations thereof. In some embodiments, cyclodextrin inclusion complexes can be used as intermediate isolation matrices to be further processed, isolated and dried (e.g., as used with waste streams).

Cyclodextrin inclusion complexes can be used to enhance the stability of the guest, convert it to a free flowing powder, or otherwise modify its solubility, delivery or performance. The amount of the guest molecule that can be encapsulated is directly related to the molecular weight of the guest molecule. In some embodiments, one mole of cyclodextrin encapsulates one mole of guest. According to this mole ratio, and by way of example only, in embodiments employing diacetyl (molecular weight of 86 Daltons) as the guest, and β-cyclodextrin (molecular weight 1135 Daltons), the maximum theoretical retention is (86/(86+1135))×100=7.04 wt %.

In some embodiments, cyclodextrin can self-assemble in solution to form a nano-structure, such as the nano-structure 20 illustrated in FIG. 2, that can incorporate three moles of a guest molecule to two moles of cyclodextrin molecules. For example, in embodiments employing diacetyl as the guest, a 10.21 wt % retention of diacetyl is possible. Other complex enhancing agents, such as pectin, can aid in the self-assembly process, and can maintain the 3:2 mole ratio of guest:cyclodextrin throughout drying. In some embodiments, because of the self-assembly of cyclodextrin molecules into nano-structures, a 5:3 mole ratio of guest:cyclodextrin is possible.

Cyclodextrin inclusion complexes form in solution. The drying process temporarily locks at least a portion of the guest in the cavity of the cyclodextrin and can produce a dry, free flowing powder.

The hydrophobic (water insoluble) nature of the cyclodextrin cavity will preferentially trap like (hydrophobic) guests most easily at the expense of more water-soluble (hydrophilic) guests. This phenomenon can result in an imbalance of components as compared to typical spray drying and a poor overall yield.

In some embodiments of the present invention, the competition between hydrophilic and hydrophobic effects is avoided by selecting key ingredients to encapsulate separately. For example, in the case of butter flavors, fatty acids and lactones form cyclodextrin inclusion complexes more easily than diacetyl. However, these compounds are not the key character impact compounds associated with butter, and they will reduce the overall yield of diacetyl and other water soluble and volatile ingredients. In some embodiments, the key ingredient in butter flavor (i.e., diacetyl) is maximized to produce a high impact, more stable, and more economical product. By way of further example, in the case of lemon flavors, most lemon flavor components will encapsulate equally well in cyclodextrin. However, terpenes (a component of lemon flavor) have little flavor value, and yet make up approximately 90% of a lemon flavor mixture, whereas citral is a key flavor ingredient for lemon flavor. In some embodiments, citral is encapsulated alone. By selecting key ingredients (e.g., diacetyl, citral, etc.) to encapsulate separately, the complexity of the starting material is reduced, allowing optimization of engineering steps and process economics.

In some embodiments, the inclusion process for forming the cyclodextrin inclusion complex is driven to completion by adding a molar excess of the guest. For example, in some embodiments, the guest is combined with the cyclodextrin in a 3:1 molar ratio of guest:cyclodextrin.

In some embodiments, the viscosity of the suspension, emulsion or mixture formed by mixing the cyclodextrin and guest molecules in a solvent is controlled, and compatibility with common spray drying technology is maintained without other adjustments, such as increasing the solids content. An emulsifier (e.g., a thickener, gelling agent, polysaccharide, hydrocolloid) can be added to maintain intimate contact between the cyclodextrin and the guest, and to aid in the inclusion process. Particularly, low molecular weight hydrocolloids can be used. One preferred hydrocolloid is pectin. Emulsifiers can aid in the inclusion process without requiring the use of high heat or co-solvents (e.g., ethanol, acetone, isopropanol, etc.) to increase solubility.

In some embodiments, the water content of the suspension, emulsion or mixture is reduced to essentially force the guest to behave as a hydrophobic compound. This process can increase the retention of even relatively hydrophilic guests, such as acetaldehyde, diacetyl, dimethyl sulfide, etc. Reducing the water content can also maximize the throughput through the spray dryer and reduce the opportunity of volatile guests blowing off in the process, which can reduce overall yield.

In some embodiments of the present invention, a cyclodextrin inclusion complex can be formed by the following process, which may include some or all of the following steps:

(1) Dry blending cyclodextrin and an emulsifier (e.g., pectin);

(2) Combining the dry blend of cyclodextrin and the emulsifier with a hot liquid or solvent such as water in a reactor, and agitating;

(3) Adding the guest and stirring (e.g., for approximately 5 to 8 hours);

(4) Cooling the reactor (e.g., turning on a cooling jacket);

(5) Stirring the mixture (e.g., for approximately 12 to 36 hours);

(6) Emulsifying (e.g., with an in-tank lightning mixer or high shear drop-in mixer); and

(7) Drying the cyclodextrin inclusion complex to form a powder.

These steps need not necessarily be performed in the order listed. In addition, the above process has proved to be very robust in that the process can be performed using variations in temperature, time of mixing, and other process parameters.

In some embodiments, step 1 in the process described above can be accomplished using an in-tank mixer in the reactor to which the hot water will be added in step 2. For example, in some embodiments, the process above is accomplished using a 1000 gallon reactor equipped with a jacket for temperature control and an inline high shear mixer, and the reactor is directly connected to a spray drier. In some embodiments, the cyclodextrin and emulsifier can be dry blended in a separate apparatus (e.g., a ribbon blender, etc.) and then added to the reactor in which the remainder of the above process is completed.

A variety of weight percents of an emulsifier to cyclodextrin can be used, including, without limitation, an emulsifier:cyclodextrin weight percent of at least about 0.5%, particularly, at least about 1%, and more particularly, at least about 2%. In addition, an emulsifier:cyclodextrin weight percent of less than about 10% can be used, particularly, less than about 6%, and more particularly, less than about 4%.

Step 2 in the process described above can be accomplished in a reactor that is jacketed for heating, cooling, or both. The reactor size can be dependent on the production size. For example, a 100 gallon reactor can be used. The reactor can include a paddle agitator and a condenser unit. In some embodiments, step 1 is completed in the reactor, and in step 2, hot deionized water is added to the dry blend of cyclodextrin and pectin in the same reactor.

Step 3 can be accomplished in a sealed reactor, or the reactor can be temporarily exposed to the environment while the guest is added, and the reactor can be re-sealed after the addition of the guest.

Step 4 can be accomplished using a coolant system that includes a cooling jacket. For example, the reactor can be cooled with a propylene glycol coolant and a cooling jacket.

The agitating in step 2, the stirring in step 3, and the stirring in step 5 can be accomplished by at least one of shaking, stirring, tumbling, and combinations thereof.

In step 6, the mixture of the cyclodextrin, emulsifier, water and guest can be emulsified using at least one of a high shear mixer (e.g., a ROSS-brand mixer at 10,000 RPM for 90 seconds), a lightning mixer, or simple mixing followed by transfer to a homogenization pump that is part of a spray dryer, and combinations thereof.

Step 7 in the process described above can be accomplished by at least one of air drying, vacuum drying, spray drying (e.g., with a nozzle spray drier, a spinning disc spray drier, etc.), oven drying, and combinations thereof.

The process outlined above can be used to provide cyclodextrin inclusion complexes with a variety of guests for a variety of applications. For example, some of the embodiments of the present invention provide a cyclodextrin inclusion complex with a guest comprising diacetyl, which can be used for various food products as a butter flavoring (e.g., in microwave popcorn, baked goods, etc.). In addition, some embodiments provide a cyclodextrin inclusion complex with a guest comprising citral, which can be used for acid stable beverages. Furthermore, some embodiments provide a cyclodextrin inclusion complex with a combination of flavor molecules as the guest that can mimic the butter flavoring of diacetyl. For example, the cyclodextrin inclusion complex can alternatively include at least one of dimethyl sulfide (a volatile sulfur compound), proline (an amino acid) and furaneol (a sweetness enhancer) as the guest. This diacetyl-free cyclodextrin inclusion complex can be used to provide a butter flavoring to food products, such as those described above.

Various features and aspects of the invention are set forth in the following examples.

### Example 1

### Cyclodextrin Inclusion Complex with β-Cyclodextrin and Diacetyl and Process for Forming Same

At atmospheric pressure, in a 100 gallon reactor, 49895.1600 g (110.02 lb) of β-cyclodextrin was dry blended with 997.9 g (2.20 lb) of beet pectin (2 wt % of pectin: β-cyclodextrin; XPQ EMP 5 beet pectin available from Degussa-France) to form a dry blend. The 100 gallon reactor was jacketed for heating and cooling, included a paddle agitator, and included a condenser unit. The reactor was supplied with a propylene glycol coolant at approximately 40° F. (4.5° C.). The propylene glycol coolant system is initially turned off, and the jacket acts somewhat as an insulator for the reactor. 124737.9 g (275.05 lb) of hot deionized water was added to the dry blend of β-cyclodextrin and pectin. The water had a temperature of approximately 118° F. (48° C.). The mixture was stirred for approximately 30 min. using the paddle agitator of the reactor. The reactor was then temporarily opened, and 11226.4110 g (24.75 lb) of diacetyl was added. The reactor was resealed, and the resulting mixture was stirred for 8 hours with no added heat. Then, the reactor jacket was connected to the propylene glycol coolant system. The coolant was turned on to approximately 40° F. (4.5° C.), and the mixture was stirred for approximately 36 hours. The mixture was then emulsified using a high shear tank mixer, such as what is typically used in spray dry operations. The mixture was then spray dried on a nozzle dryer having an inlet temperature of approximately 410° F. (210° C.) and an outlet temperature of approximately 221° F. (105° C.). A percent retention of 18.37 wt % of diacetyl in the cyclodextrin inclusion complex was achieved. The moisture content was measured at 4.0%. The cyclodextrin inclusion complex included less than 0.3% surface diacetyl, and the particle size of the cyclodextrin inclusion complex was measured as 99.7% through an 80 mesh screen.

### Example 2

### Cyclodextrin Inclusion Complex with α-Cyclodextrin and Diacetyl and Process for Forming Same

The β-cyclodextrin of example 1 was replaced with a-cyclodextrin and dry blended with 1 wt % pectin (i.e., 1 wt % of pectin: α-cyclodextrin; XPQ EMP 5 beet pectin available from Degussa-France). The mixture was processed and dried by the method set forth in Example 1. The percent retention of diacetyl in the cyclodextrin inclusion complex was 11.4 wt %.

### Example 3

### Cyclodextrin Inclusion Complex with β-Cyclodextrin and Orange Essence and Process for Forming SAME

Orange essence, an aqueous waste stream from juice production, was added as the aqueous phase to a dry blend of β-cyclodextrin and 2 wt % pectin, formed according to the process set forth in Example 1. No additional water was added, the solids content was approximately 28%. The cyclodextrin inclusion complex was formed by the method set forth in Example 1. The dry inclusion complex contained approximately 3 to 4 wt % acetaldehyde, approximately 5 to 7 wt % ethyl butyrate, approximately 2 to 3 wt % linalool and other citrus enhancing notes. The resulting cyclodextrin inclusion complex can be useful in top-noting beverages.

### Example 4

### Cyclodextrin Inclusion Complex with β-Cyclodextrin and Acetyl Propionyl and Process for Forming Same

A molar excess of acetyl propionyl was added to a dry blend of β-cyclodextrin and 2 wt % pectin in water, following the method set forth in Example 1. The percent retention of acetyl propionyl in the cyclodextrin inclusion complex was 9.27 wt %. The mixture can be useful in top-noting diacetyl-free butter systems.

### Example 5

### Orange Oil Flavor Product and Process for Forming Same

Orange oil (i.e., Orange Bresil; 75 g) was added to an aqueous phase comprising 635 g of water, 403.75 g of maltodextrin, and 21.25 g of beet pectin (available from Degussa—France, product no. XPQ EMP 5). The orange oil was added to the aqueous phase with gentle stirring, followed by strong stirring at 10,000 RPM to form a mixture. The mixture was then passed through a homogenizer at 250 bars to form an emulsion. The emulsion was dried using a NIRO-brand spray drier having an inlet temperature of approximately 180° C. and an outlet temperature of approximately 90° C. to form a dried product. The percent flavor retention was then quantified as the amount of oil (in g) in 100 g of the dried product, divided by the oil content in the starting mixture. The percent retention of orange oil was approximately 91.5%.

### Example 6

### Orange Oil Flavor Product and Process for Forming Same

Orange oil (75 g) was added to an aqueous phase comprising 635 g of water, 297.50 g of maltodextrin, and 127.50 g gum arabic (available from Colloids Naturels International). The orange oil was added to the aqueous phase and dried following the method set forth in Example 5. The percent flavor retention was approximately 91.5%.

### Example 7

### Orange Oil Flavor Product and Process for Forming Same

Orange oil (75 g) was added to an aqueous phase comprising 635 g of water, 297.50 g of maltodextrin, 123.25 g gum arabic (available from Colloids Naturels International), and 4.25 g of depolymerized citrus pectin. The orange oil was added to the aqueous phase and dried following the method set forth in Example 5. The percent flavor retention was approximately 96.9%.

### Example 8

### Orange Oil Flavor Product and Process for Forming Same

Orange oil (75 g) was added to an aqueous phase comprising 635 g of water, 297.50 g of maltodextrin, 123.25 g gum arabic (available from Colloids Naturels International), and 4.25 g of beet pectin (available from Degussa—France, product no. XPQ EMP 5). The orange oil was added to the aqueous phase and dried following the method set forth in Example 5. The percent flavor retention was approximately 99.0%.

### Example 9

### Orange Oil Flavor Product and Process for Forming Same

Orange oil (75 g) was added to an aqueous phase comprising 635 g of water, 403.75 g of maltodextrin, and 21.25 g of depolymerized citrus pectin. The orange oil was added to the aqueous phase and dried following the method set forth in Example 5. The percent flavor retention was approximately 90.0%.

### Example 10

### Orange Oil Flavor Product and Process for Forming Same

Orange oil (75 g) was added to an aqueous phase comprising 635 g of water, 340.00 g of maltodextrin, and 85.00 g gum arabic (available from Colloids Naturels International). The orange oil was added to the aqueous phase and dried following the method set forth in Example 5. The percent flavor retention was approximately 91.0%.

### Example 11

### Orange Oil Flavor Product and Process for Forming Same

Orange oil (75 g) was added to an aqueous phase comprising 635 g of water and 425.00 g of maltodextrin. The orange oil was added to the aqueous phase and dried following the method set forth in Example 5. The percent flavor retention was approximately 61.0%.

### Example 12

### Orange Oil Flavor Product and Process for Forming Same

Orange oil (75 g) was added to an aqueous phase comprising 635 g of water, 420.75 g of maltodextrin, and 4.25 g of pectin. The orange oil was added to the aqueous phase and dried following the method set forth in Example 5. The percent flavor retention was approximately 61.9%.

### Example 13

### Orange Oil Flavor Product and Process for Forming Same

Orange oil (75 g) was added to an aqueous phase comprising 635 g of water, 403.75 g of maltodextrin, and 21.50 g of pectin. The orange oil was added to the aqueous phase and dried following the method set forth in Example 5. The percent flavor retention was approximately 71.5%.

### Example 14

### Orange Oil Flavor Product and Process for Forming Same

Orange oil (75 g) was added to an aqueous phase comprising 635 g of water, 420.75 g of maltodextrin, and 4.75 g of depolymerized citrus pectin. The orange oil was added to the aqueous phase and dried following the method set forth in Example 5. The percent flavor retention was approximately 72.5%.

### Example 15

### Orange Oil Flavor Product and Process for Forming Same

Orange oil (75 g) was added to an aqueous phase comprising 635 g of water, 420.75 g of maltodextrin, and 4.75 g of beet pectin (available from Degussa-France, product no. XPQ EMP 5). The orange oil was added to the aqueous phase and dried following the method set forth in Example 5. The percent flavor retention was approximately 78.0%.

### Example 16

### Orange Oil Flavor Product and Process for Forming Same

Orange oil (75 g) was added to an aqueous phase comprising 635 g of water, 414.40 g of maltodextrin, and 10.60 g of depolymerized citrus pectin. The orange oil was added to the aqueous phase and dried following the method set forth in Example 5. The percent flavor retention was approximately 85.0%.

### Example 17

### Orange Oil Flavor Product and Process for Forming Same

Orange oil (75 g) was added to an aqueous phase comprising 635 g of water, 414.40 g of maltodextrin, and 10.60 g of beet pectin (available from Degussa-France, product no. XPQ EMP 5). The orange oil was added to the aqueous phase and dried following the method set forth in Example 5. The percent flavor retention was approximately 87.0%.

Various features and aspects of the invention are set forth in the following claims.

1. A method for preparing a cyclodextrin inclusion complex, the method comprising:
dry blending cyclodextrin and an emulsifier to form a dry blend; and
combining a solvent and a guest with the dry blend to form a cyclodextrin inclusion complex.

2. The method of claim 1, wherein combining the solvent and the guest with the dry blend to form a cyclodextrin inclusion complex forms a mixture comprising the cyclodextrin inclusion complex, and the method further comprises drying the mixture.

3. The method of claim 2, wherein drying includes at least one of air drying, vacuum drying, spray drying, oven drying, and a combination thereof.

4. The method of claim 2, further comprising emulsifying the mixture with at least one of an in-tank mixer and a high shear drop-in mixer prior to drying.

5. The method of claim 2, wherein the mixture includes a solvent content, and the method further comprises reducing the solvent content of the mixture prior to drying the mixture.

6. The method of claim 1, wherein combining the guest and the solvent with the dry blend includes combining the solvent and the dry blend, and then combining the guest therewith.

7. The method of claim 1, wherein the emulsifier comprises a hydrocolloid.

8. The method of claim 1, wherein the emulsifier comprises at least one of xanthan gum, pectin, gum acacia, tragacanth, guar, carrageenan, locust bean, and a combination thereof.

9. The method of claim 1, wherein the emulsifier comprises pectin.

10. The method of claim 9, wherein the pectin includes at least one of beet pectin, fruit pectin, and a combination thereof.

11. The method of claim 1, wherein the solvent comprises water.

12. The method of claim 1, wherein the cyclodextrin includes at least one of α-cyclodextrin, β-cyclodextrin, γ-cyclodextrin, and a combination thereof.

13. The method of claim 1, wherein the guest includes at least one of a flavor, an olfactant, a pharmaceutical agent, a nutraceutical agent, and a combination thereof.

14. The method of claim 13, wherein the flavor includes at least one of an aldehyde, a ketone, an alcohol, and a combination thereof.

15. The method of claim 13, wherein the olfactant includes at least one of natural fragrances, synthetic fragrances, synthetic essential oils, natural essential oils, and a combination thereof.

16. The method of claim 1, wherein the guest includes at least one of fatty acids, lactones, terpenes, diacetyl, dimethyl sulfide, proline, furaneol, linalool, acetyl propionyl, natural essences, essential oils, and a combination thereof.

17. The method of claim 1, wherein the guest includes diacetyl.

18. The method of claim 1, wherein the cyclodextrin inclusion complex is at least partially defined by a nano-structure comprising the cyclodextrin and guest.

19. The method of claim 18, wherein the nano-structure includes a mole ratio of guest:cyclodextrin of at least approximately 3:2.

20. The method of claim 1, wherein combining a guest and a solvent with the dry blend includes combining the guest and cyclodextrin in a molar ratio of guest:cyclodextlin of approximately 3:1.

21. The method of claim 1, wherein the cyclodextrin inclusion complex is formed without additional heat or co-solvents.

22. The method of claim 1, wherein combining the guest and the solvent with the dry blend includes stirring the guest, the solvent and the dry blend.

23. The method of claim 22, wherein stirring the guest, the dry blend and the solvent occurs for approximately 5 to 8 hours.

24. The method of claim 1, wherein combining a solvent and a guest with the dry blend to form a cyclodextrin inclusion complex includes forming a mixture comprising a cyclodextrin inclusion complex, and the method further comprises cooling the mixture.

25. The method of claim 24, wherein cooling the mixture includes turning on a cooling jacket of a reactor.

26. The method of claim 1, wherein combining a guest and a solvent with the dry blend is performed in a sealed reactor.

27. The method of claim 1, wherein dry blending cyclodextrin and an emulsifier includes dry blending cyclodextrin and an emulsifier in a weight percent of emulsifier:cyclodextrin of at least about 0.5 wt %.

28. The method of claim 1, wherein dry blending cyclodextrin and an emulsifier includes dry blending cyclodextrin and an emulsifier in a weight percent of emulsifier:cyclodextrin of less than about 10 wt %.

29. A cyclodextrin inclusion complex prepared according to the method of claim 1.

30. The cyclodextrin inclusion complex of claim 29, wherein the weight percent of guest to cyclodextrin in the cyclodextrin inclusion complex is at least approximately 10 wt %.

31. The cyclodextrin inclusion complex of claim 29, wherein the cyclodextrin inclusion complex is used in at least one of foods, chewing gums, candy, flavorings, fragrances, pharmaceuticals, nutraceuticals, cosmetics, agricultural applications, photographic emulsions, waste stream systems, and a combination thereof.

32. A method for preparing a cyclodextrin inclusion complex, the method comprising:
combining cyclodextrin and an emulsifier to form a first mixture;
combining the first mixture with a solvent to form a second mixture; and
combining a guest with the second mixture to form a third mixture.

33. The method of claim 32, further comprising drying the third mixture.

34. The method of claim 32, wherein combining cyclodextrin and an emulsifier includes dry blending.

35. The method of claim 32, wherein the emulsifier comprises at least one of xanthan gum, pectin, gum acacia, tragacanth, guar, carrageenan, locust bean, and a combination thereof.

36. The method of claim 32, wherein the emulsifier comprises pectin.

37. The method of claim 32, wherein the guest comprises at least one of a flavor, an olfactant, a pharmaceutical agent, a nutraceutical agent, and a combination thereof.

38. The method of claim 32, wherein the guest comprises diacetyl.

39. A cyclodextrin inclusion complex prepared according to the method of claim 32.

40. A method for preparing a cyclodextrin inclusion complex, the method comprising:
dry blending cyclodextrin and pectin to form a first mixture;
combining the first mixture with water to form a second mixture; and
combining diacetyl with the second mixture to form a third mixture.

41. The method of claim 40, further comprising drying the third mixture.

42. The method of claim 40, wherein the cyclodextrin and pectin are dry blended in a weight percent of pectin:cyclodextrin of at least approximately 0.5 wt %.

43. The method of claim 40, wherein the cyclodextrin and pectin are dry blended in a weight percent of pectin:cyclodextrin of less than approximately 10 wt %.

44. A cyclodextrin inclusion complex prepared according to the method of claim 40.
